(19) United States
(12) Patent Application Publication
(10) Pub. No.: US 2025/0241989 A1
(43) Pub. Date: Jul. 31, 2025

(54) Neuregulin for Protection Against Respiratory Viral Infection and Post-Viral Disease

(71) Applicant: THE RESEARCH INSTITUTE AT NATIONWIDE CHILDREN'S HOSPITAL, Columbus, OH (US)

(72) Inventors: Mitchell GRAYSON, COLUMBUS, OH (US); Syed-Rehan HUSSAIN, COLUMBUS, OH (US)

(21) Appl. No.: 18/703,595

(22) PCT Filed: Oct. 21, 2022
(86) PCT No.: PCT/US2022/078494

§ 371 Date: Apr. 22, 2024

(51) Int. Cl. A61K 38/18; A61K 9/00; A61P 31/14

(52) U.S. Cl. CPC A61K 38/1883; A61K 9/0043; A61P 31/14

(57) ABSTRACT

A method of treating or decreasing the risk of developing a respiratory viral infection in a subject is described. The method includes administering a therapeutically effective amount of neuregulin to the subject. A method of decreasing the risk that a subject will develop post-viral airway disease by administering an effective amount of neuregulin to the subject is also described.

### CROSS-REFERENCE TO RELATED APPLICATIONS

This application claims priority to U.S. Provisional Application Ser. No. 63/270,624, filed on Oct. 22, 2021, which is hereby incorporated by reference in its entirety.

### SEQUENCE LISTING

The instant application contains a Sequence Listing which has been submitted in ASCII format via EFS-Web and is hereby incorporated by reference in its entirety.

### BACKGROUND

Respiratory viral infections with negative-strand RNA viruses, such as influenza virus (IAV), respiratory syncytial virus (RSV) and parainfluenza virus (PIV) are a major cause of morbidity and mortality. Several groups have demonstrated pre-existing atopy protects from mortality to IAV in mouse models (Ishikawa et al., J Clin Immunol 32:256-267 (2012)); (An et al., Front Immunol 9:986 (2018)); (Furuya et al., PLoS Pathog 11: e1005180 (2015)). Human data supports this relationship; in the 2009 H1N1 IAV pandemic patients with asthma hospitalized with IAV had less severe outcomes, a recent meta-analysis concluded patients with asthma had a significantly lower mortality to COVID-19, and the presence of food allergies appeared to have reduced the likelihood of being infected with SARS-COV-2 (Hou et al., Clin Immunol Pract 9:3944-3968 e3945 (2021)). The mechanistic explanation for why pre-existing atopy would be protective is not known, though various explanations have been provided for the increased survival in animal models including the activation of NK cells, induction of Type III interferon and expression of TGFβ.

Respiratory syncytial virus (RSV) is a major viral pathogen, especially for infants and the elderly. Annually there are on average 2.1 million outpatient visits and 58,000 hospitalizations for RSV in children under 5 years of age. Severe infection with RSV in infancy is associated with a markedly increased risk of developing asthma and atopic disease. In those 65 years of age or older, RSV accounts for an average of 177,000 hospitalizations and 14,000 deaths annually, with similar mortality rates to influenza (IAV). Reducing severity of an RSV infection has potential for significant clinical impact by reducing mortality in adults and preventing induction of atopy in infants.

### SUMMARY OF THE INVENTION

The inventors have developed a high-fidelity model of respiratory viral infection. Their mouse model utilizes a natural rodent pathogen, Sendai virus (SeV), which is closely related to RSV, but unlike RSV, faithfully replicates in mouse airway epithelial cells. Infection with SeV leads to acute bronchiolitis followed by a chronic inflammatory response associated with airway hyper-reactivity and mucous cell metaplasia. These are the cardinal features of human paramyxoviral (PIV), pneumoviral (RSV), and orthomyxoviral (Influenza A) infection, and the inventors have used this model to identify a novel immune axis, which we have validated in humans: (Khan and Grayson, J Allergy Clin Immunol 125:265-267 (2010)); (Sammon et al., Ann Allergy Asthma Immunol 123:508-511 e501 (2019)).

The inventors recently defined a mechanistic pathway involving neutrophils that explains how pre-existing atopy prevents development of post-viral airway disease (Hussain et al., J Immunol 207:2589-2597 (2021)). Further, like previous studies with IAV infection in atopic mice, in their model they found that pre-existing atopy protects from mortality against SeV. Herein, the inventors have provided data demonstrating that this protection does not depend upon neutrophils, but does depend upon CD11c expressing cells that over-express Neuregulin 1 (NRG1), a cytokine of the epidermal growth factor family, that interacts with ErbB receptor tyrosine kinases. Moreover, exogenously administered NRG1 in non-atopic mice significantly reduced mortality to the viral insult. Finally, the inventors demonstrate that in vitro NRG1 treatment can reduce SeV replication in well-differentiated mouse tracheal epithelial cells and, more importantly, RSV replication in human bronchial epithelial cells. The studies demonstrate that NRG1 protects from lethal respiratory viral infection, and may be the mechanism by which pre-existing atopy protects against lethal respiratory viral infection.

Severe respiratory viral infections are associated with significant mortality in infants and the elderly; however, allergic disease can protect from these outcomes. Work by the inventors identified a protein called neuregulin-1 (NRG1), produced by cells of the immune system in allergic mice, that provides a survival advantage against respiratory viral infection. NRG1 pretreatment in non-atopic mice infected with a lethal dose of a rodent RNA virus (Sendai virus), similar to human Respiratory Syncytial Virus, significantly reduced death. Further, NRG1 pretreatment reduced viral replication in human and mouse airway epithelial cell cultures. These studies signify a potential therapeutic role of NRG1 in modulating the severity of respiratory viral infections.

### BRIEF DESCRIPTION OF THE FIGURES

The present invention may be more readily understood by reference to the following figures, wherein:

FIGS. 1A and 1B provide graphs showing atopy prevents lethal SeV infection. (A) Mice sensitized and challenged with house dust mite extract (“H/H/SeV”, atopic) are protected from mortality to high dose (2×10⁶ pfu) SeV infection, while non-atopic (NA) mice (i.e., only sensitized (“H/P/SeV”) or neither sensitized or challenged (“P/P/SeV”)) all succumbed to the viral insult (p=0.0027 H/H/SeV versus each of the other NA groups, Mantel-Cox). (B) Peak SeV titers (day 5 PI; both at regular (2×10⁵ pfu) and high dose inoculation) are reduced in atopic mice compared to NA mice. n≥3 per group.

FIGS. 2A-2D provide graphs showing CD11c⁺ cells in atopic mice delay mortality to SeV and produce neuregulin-1 (NRG1). (A) CD11c⁺ cells are increased in the lungs of atopic mice. Flow cytometry of lung cell suspension showing frequency (left) and cell number (right) at day 3 PI high dose SeV (B) Adoptive transfer of CD11c+ cells isolated from NA or atopic mice into naïve mice 24 h before inoculation with high dose SeV delays but does not prevent mortality; n=4 per group. (C) Transcriptomic (RNAseq) comparison between FACS isolated lung CD11c⁺ cells from atopic and NA mice identifies several disparately expressed gene products, including Nrg1 (n=4 per group). Selected gene products shown. (D) NRG1 is markedly increased in atopic mouse lung (“tissue”), BAL, and supernatant from 1×10⁶ atopic lung CD11c⁺ cells (“CD11c sup”) cultured for 24 h. *p<0.05, **p<0.01, ****p<0.0001

FIGS. 3A & 3B provide graphs showing NRG1 is sufficient to reduce mortality to SeV. NRG1 (1 ng to 1000 ng) i.n. (in 30 μL) given daily to naïve mice for 5 d before inoculation with high dose SeV reduces viral mortality; n=4 per group (1 ng, 10 ng 1000 ng & PBS) n=8 per group (100 ng and 500 ng). (B) Survival of CD11c/ROSA-iDTR or littermate (LM) mice with or without NRG1 treatment (daily i.n. from day −5 to day 0) and 15 ng/gm diphtheria toxin (DTx) (i.p. on day −2) before inoculation with 2×10⁵ pfu (regular dose) SeV on day 0. n≥4 per group. DTx treatment depletes CD11c⁺ cells in CD11c/ROSA-iDTR but not LM mice.

FIGS. 4A-4D provide graphs and images showing NRG1 treatment reduces viral replication and regulates gene expression in airway epithelium. (A) Adding NRG1 to human bronchial epithelial cells (hBEC) inoculated with recombinant GFP expressing RSV (rgRSV) (left) and mouse tracheal epithelial cells (mTEC) inoculated with GFP expressing SeV (GFP-SeV; right) reduces spread of infection. Representative images shown. (B) Quantification of (A) for rgRSV and hBEC and (C) for GFP-SeV and mTEC. GFP positive cells quantified by ImageJ. Representative images from ≥3 separate experiments. *p<0.05, **p<0.01. (D) Transcriptomic analysis of hBEC cultures treated with NRG1 (100 ng) on the basolateral side of the Transwell for 5 days and inoculated with RSV (4000 pfu). RNA was isolated 48 h PI RSV and qRT-PCR performed using a custom Prime PCR array plate: (i) Transcripts in which NRG1 treatment reduced gene expression from that seen in RSV infected cells. (ii) Transcripts with low level expression that show small but significant change in expression relative to naïve control with NRG1 alone or genes significantly increased with RSV but whose expression levels were not affected by NRG1. *p<0.05, **p<0.01, ***p<0.001, ****p<0.0001, n=3.

FIGS. 5A-5D provide graphs showing Alarmins induce CD11c⁺ cells to produce NRG1. CD11c⁺ cells from naïve mouse lung cultured with (A) IL33 or (B) Thymic stromal lymphopoietin (TSLP) and supernatant NRG1 determined by ELISA 24 h later; samples run in duplicate. (C) NRG1 determined by ELISA (R&D Systems #DY377) of supernatant from peripheral blood human CD14+ monocytes (negative immunomagnetic selection) cultured for 18 h with or without IL33 or TSLP. (D) Naïve B6 mice given IL33 (1.5 μg) i.n. for 5 d before SeV infection had significantly reduced mortality compared to PBS treated mice; n≥4 per group.

### DETAILED DESCRIPTION OF THE INVENTION

The present invention provides a method of treating or decreasing the risk of severe outcomes from a respiratory viral infection in a subject. The method includes administering a therapeutically effective amount of neuregulin to the subject. A method of decreasing the risk that a subject will develop post-viral airway disease by administering an effective amount of neuregulin to the subject is also provided.

### Definitions

As used herein, the term “diagnosis” can encompass determining the likelihood that a subject will develop a disease, or the existence or nature of disease in a subject. The term diagnosis, as used herein also encompasses determining the severity and probable outcome of disease or episode of disease or prospect of recovery, which is generally referred to as prognosis).

As used herein, the terms “treatment,” “treating,” and the like, refer to obtaining a desired pharmacologic or physiologic effect. The effect may be therapeutic in terms of a partial or complete cure for a disease or an adverse effect attributable to the disease. “Treatment,” as used herein, covers any treatment of a disease in a mammal, particularly in a human, and can include inhibiting the disease or condition, i.e., arresting its development; and relieving the disease, i.e., causing regression of the disease.

As used herein, the term “preventing” includes preventing the onset of a clinically evident disease (e.g., viral infection) altogether, preventing the onset of a preclinically evident stage of disease (e.g., viral infection) in a subject, or decreasing the risk that a subject will develop clinically evident disease. Preventative treatment can be particularly useful in subjects identified as having an elevated risk of developing a viral infection. An elevated risk represents an above-average risk that a subject will develop a viral infection. Examples of elevated risk include exposure to viral infection, residence in a hospital, or a pre-existing condition that increases the risk of developing a viral infection such as diabetes or immunosuppression.

The terms “therapeutically effective” and “pharmacologically effective” are intended to qualify the amount of an agent which will achieve the goal of improvement in disease severity and the frequency of incidence over treatment of each agent by itself, while avoiding adverse side effects typically associated with alternative therapies. The effectiveness of treatment may be measured by evaluating a reduction in symptoms.

The terms “polypeptide” and “peptide” as used herein, are used interchangeably and refer to a polymer of amino acids. These terms do not connote a specific length of a polymer of amino acids. Thus, for example, the terms oligopeptide, protein, and enzyme are included within the definition of polypeptide or peptide, whether produced using recombinant techniques, chemical or enzymatic synthesis, or naturally occurring. This term also includes polypeptides that have been modified or derivatized, such as by glycosylation, acetylation, phosphorylation, and the like.

The terms “subject” and “patient” can be used interchangeably herein, and generally refer to a mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos. Treatment and evaluation of human subjects is of particular interest. Human subjects can be various ages, such as a child (under 18 years), adult (18 to 59 years) or elderly (60 years or older) human subject.

Unless defined otherwise, all technical and scientific terms used herein have the same meaning as commonly understood by one of ordinary skill in the art to which this invention belongs.

As used herein and in the appended claims, the singular forms “a”, “and”, and “the” include plural referents unless the context clearly dictates otherwise. Thus, for example, reference to “a sample” also includes a plurality of such samples and reference to “a biomarker” includes reference to one or more biomarkers, and so forth.

### Treating or Preventing Respiratory Viral Infections

In one aspect, the present invention provides a method of treating or decreasing the risk of developing a respiratory viral infection in a subject by administering a therapeutically effective amount of neuregulin to the subject. In some embodiments, the method decreases the risk of developing a respiratory viral infection, while in other embodiments the method treats an existing respiratory viral infection. Treatment includes decreasing the severity of an infection, and in some cases reducing the severity to the point of rendering the infection asymptomatic.

### Neuregulins (NRG)

Neuregulins are a family of four structurally related proteins that are part of the EGF family of proteins, and include neuregulin 1, 2, 3, and 4. Neuregulin-1 (NRG-1) exists as 14 isoforms due to alternate splicing or use of alternate promoters. Examples of different NRG-1 isoforms include type I (Heregulin), type II (Glial Growth Factor-2), type III (Sensory and motor neuron-derived factor), type IV, type V, and type VI NRG-1. All of the isoforms contain epidermal growth factor (EGF-like) domain, either alpha or beta variant that differs in the C-terminal, that are required for the direct binding to ErbB (erythroblastic oncogene B) receptor tyrosine kinases. The EGF-like motif is sufficient for most of the biological effects of the full-length protein.

All NRG1 isoforms share a conserved EGF-like domain that interacts with the ErbB family of tyrosine kinase receptors to induce signaling. This EGF-like motif is capable of mimicking most of the biological effects of full-length protein. However, other structural extracellular domains that distinguishes NRG-1 isoforms (type I-III) may contribute to specific biological functions in various cell types. the EGF domain of NRG1 and NRG2 exist as alpha and beta form. (Buonanno A, Fischbach G D, Curr Opinion in Neurobiol., 11 (3): 287-96 (2001)), the disclosure of which is incorporated by reference herein. Buonanno & Fischbach also provide a sequence homology comparison between various neuregulin proteins, showing the substantial similarity of these sequences.

The inventors have used recombinant human and mouse NRG-1 alpha and shown that the viral replication is significantly reduced in NRG-1-treated pseudostratified well-differentiated airway epithelial cells [human (hBEC), mouse (mTEC)] when they were infected with RSV and Sendai virus (SeV) respectively. Furthermore, in vivo delivery of NRG-1 to mouse significantly reduced mortality with normally lethal dose (2.00E+06 pfu) of SeV. NRG-1 beta is also expected to provide protection against respiratory viral infection, since NRG-1 beta has been reported to have stronger binding affinity to the ErbB receptors. There are three other structurally related neuregulin family members (NRG-2, NRG-3, NRG-4) that can also provide protection against viral infection. In some embodiments, the neuregulin administered to the subject is neuregulin-1.

In some embodiments, recombinant human neuregulin-1 alpha (NRG1 alpha) is used. NRG1 alpha is a single, non-glycosylated polypeptide chain, and is commercially available from, for example, NovusBio™. NRG1 alpha is assigned Accession No. KAI2549591, and has the amino acid sequence SHLVKCAEKEKTFCVNGGECFMVKDLSNPSRYLCKCQPGFTGARCTENVPMKVQN QEKAEELYQK SEQ ID NO: 1). Accordingly, in some embodiments, a sequence substantially similar to SEQ ID NO: 1 can be used. A substantially similar sequence can be 85%, 90%, or 95% identical to SEQ ID NO: 1, with only conservative amino acid substitutions.

Functional-conservative derivatives or variants may result from modifications and changes that may be made in the structure of a polypeptide (and in the DNA sequence encoding it), and still obtain a functional molecule with desirable characteristics (e.g., antiviral effects). Functional-conservative derivatives may also consist of a fragment of a polypeptide that retains its functionality.

Accordingly, functional-conservative derivatives or variants are those in which a given amino acid residue in a protein has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A functional-conservative derivative also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared. Two amino acid sequences are “substantially homologous” or “substantially similar” when greater than 80%, preferably greater than 85%, preferably greater than 90% of the amino acids are identical, or greater than about 90%, preferably greater than 95%, are similar (functionally identical). Preferably, the similar or homologous sequences are identified by alignment using, for example, the GCG (Genetics Computer Group, Program Manual for the GCG Package, Version 7, Madison, Wisconsin) pileup program, or any of sequence comparison algorithms such as BLAST, FASTA, etc.

### Respiratory Viral Infection

Respiratory viral infection is an infection of the respiratory tract by a virus (e.g., a respiratory virus). A number of respiratory viruses are known to those skilled in the art. See H. F. Boncristiani, “Respiratory Viruses,” Encyclopedia of Microbiology, 500-518 (2009). In some embodiments, the respiratory virus is a negative-strand RNA viruses, such as influenza virus, respiratory syncytial virus, or parainfluenza virus. Common respiratory viruses include influenza virus, respiratory syncytial virus, parainfluenza viruses, metapneumovirus, rhinovirus, coronaviruses, adenoviruses, and bocaviruses. In some embodiments, the respiratory viral infection is a respiratory syncytial virus or parainfluenza virus infection.

In some embodiments, a subject being treated for an existing respiratory viral infection has been diagnosed as having a respiratory viral infection. Respiratory tract infections include upper respiratory tract infections and lower respiratory tract infections, which lower respiratory tract infections such as pneumonia and bronchitis tending to be more severe. The upper respiratory track includes the airway above the vocal cords, and includes the nose, sinuses, pharynx, and larynx. The lower respiratory tract comprises the trachea, bronchial tubes, bronchioles, and the lungs. PCR analysis and a review of a patient's clinical history, or pulmonary functional testing can be used to diagnose a variety of different respiratory tract infections. Zavorsky, G. S., Respir Physiol Neurobiol., 186 (1): 103-8 (2013)

A subject being administered neuregulin to treat or prevent respiratory viral infection can also be provided with other additional antiviral therapy to treat or prevent a respiratory viral infection. Examples of methods to treat or prevent respiratory viral infection include administration of monoclonal antibodies such as palivizumab, vaccines, and administration of antiviral agents, including siRNA, small molecules (e.g., lumicitabine), nanobodies (e.g., ALX-0171) and fusion inhibitors (e.g., presatovir). The additional antiviral therapy can be provided before, simultaneously, or after neuregulin administration.

### Decreasing the Risk of Developing Post-Viral Airway Disease

A further aspect of the invention provides a method of decreasing the risk a subject will develop post-viral airway disease by administering an effective amount of neuregulin to the subject. In some embodiments, the neuregulin administered to the subject is neuregulin-1. In some embodiments, the subject has a respiratory virus infection, while in further embodiments the respiratory virus infection is a respiratory syncytial virus (RSV) or parainfluenza virus infection. In yet further embodiments, the subject does not currently have a respiratory virus, but has an increased risk of developing a respiratory virus infection.

Decreasing the risk that a subject will develop post-viral airway disease can in some embodiments prevent the development of post-viral airway disease, which represents decreasing the risk by 100%. In other embodiments, the risk is not completely eliminated, but rather is decreased. The decrease can represent an absolute value for subjects who have developed a viral infection, or a comparative value between subjects who have received neuregulin and those who haven't after developing a viral infection. For example, the risk of developing post-viral airway disease can include about a 5% decrease, about a 10% decrease, or about a 20%, 30%, 40%, 50%, 60%, 70%, 80%, 90%, or 100% decrease. Whether or not a subject has developed a post-viral airway disease can be evaluated using the standard diagnostic methods for airway disease, such as X-ray or pulmonary function testing.

Epidemiologic data suggest that RSV infection drives an increased risk of asthma only in those without preexisting atopy. Further, in the 2009 IAV pandemic, while asthma was a risk factor for hospitalization, those with asthma hospitalized with IAV had lower mortality and decreased risk of ICU stay or need for mechanical ventilation. There are many potential explanations for these outcomes; however, recent publications have shown a protective effect of pre-existing atopy in mouse IAV models, although the mechanism(s) of this protection remains unknown. Therefore, the inventors have investigated and determined that atopy offers protection in severe respiratory viral infections.

A variety of post-viral airway diseases are known. Post-viral airway diseases are diseases involving the airway that develop after, and as a result of a respiratory viral infection. See Hussain et al., Expert Rev Clin Immunol., 15 (1): 49-58 (2019). Examples of post-viral airway diseases include asthma, postviral bronchial hyperreactivity syndrome, wheezing, and atopic disease. In some embodiments, the post-viral airway disease is asthma.

### Administration and Formulation

The invention also provides pharmaceutical compositions that can be used for the administration of active agents used in the method of the invention to a subject in need thereof. The pharmaceutical acceptable carrier can have many forms, including tablets, hard or soft gelatin capsules, aqueous solutions, suspensions, and liposomes and other slow-release formulations, such as shaped polymeric gels. An oral dosage form may be formulated such that the polypeptide or antibody is released into the intestine after passing through the stomach. Such formulations are described in U.S. Pat. No. 6,306,434 and in the references contained therein.

Oral liquid pharmaceutical compositions may be in the form of, for example, aqueous or oily suspensions, solutions, emulsions, syrups or elixirs, or may be presented as a dry product for constitution with water or other suitable vehicle before use. Such liquid pharmaceutical compositions may contain conventional additives such as suspending agents, emulsifying agents, non-aqueous vehicles (which may include edible oils), or preservatives.

The active agents can be formulated for parenteral administration (e.g., by injection, for example, bolus injection or continuous infusion) and may be presented in unit dosage form in ampules, prefilled syringes, small volume infusion containers or multi-dose containers with an added preservative. The pharmaceutical compositions may take such forms as suspensions, solutions, or emulsions in oily or aqueous vehicles, and may contain formulatory agents such as suspending, stabilizing and/or dispersing agents. Pharmaceutical compositions suitable for rectal administration can be prepared as unit dose suppositories. Suitable carriers include saline solution and other materials commonly used in the art.

In some embodiments, the neuregulin is administered by pulmonary administration (e.g., intranasal). For administration by inhalation, active agents can be conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the active agents may take the form of a dry powder composition, for example, a powder mix of a modulator and a suitable powder base such as lactose or starch. The powder composition may be presented in unit dosage form in, for example, capsules or cartridges or, e.g., gelatin or blister packs from which the powder may be administered with the aid of an inhalator or insufflator. For intra-nasal administration, the active agents may be administered via a liquid spray, such as via a plastic bottle atomizer.

Active agents can be formulated for transdermal administration. The active agents can also be formulated as an aqueous solution, suspension or dispersion, an aqueous gel, a water-in-oil emulsion, or an oil-in-water emulsion. A transdermal formulation may also be prepared by encapsulation of an active agent within a polymer, such as those described in U.S. Pat. No. 6,365,146. The dosage form may be applied directly to the skin as a lotion, cream, salve, or through use of a patch. Examples of patches that may be used for transdermal administration are described in U.S. Pat. Nos. 5,560,922 and 5,788,983.

It will be appreciated that the amount of active agent required for use in treatment will vary not only with the particular carrier selected but also with the route of administration, the nature of the condition being treated and the age and condition of the patient. Ultimately the attendant health care provider may determine proper dosage. In addition, a pharmaceutical composition may be formulated as a single unit dosage form, or it may be administered in multiple doses.

The amount of active agent that is delivered to the subject will depend upon the nature and severity of the condition being treated, and on the nature of prior treatments which the subject has undergone. Ultimately, the attending physician will decide the amount of active agent with which to treat each individual patient. For example, the attending physician can administer low doses of the active agent of the present invention and observe the patient's response. Larger doses of the active agent may be administered until the optimal therapeutic effect is obtained for the patient, and at that point the dosage is not increased further. It is contemplated that the various pharmaceutical compositions used to practice the method of the present invention should contain about 0.01 ng to about 100 mg (preferably about 0.1 μg to about 10 mg, more preferably about 0.1 μg to about 1 mg) of active agent per kg body weight

An example has been included to more clearly describe particular embodiments of the invention. However, there are a wide variety of other embodiments within the scope of the present invention, which should not be limited to the particular example provided herein.

### EXAMPLE

### Example 1: Neuregulin Protects Against Respiratory Viral Induced Mortality

We have a high-fidelity mouse model of post-viral airway disease that utilizes a natural rodent pathogen, Sendai virus (SeV) similar to RSV, which faithfully replicates in mouse airway epithelial cells causing acute bronchiolitis followed by airway hyperreactivity due to chronic inflammation. In this report we show that making mice atopic with house dust mite extract before viral infection prevents mortality to a normally lethal dose of SeV. Moreover, CD11c⁺ cells in atopic mice provided some survival advantage by delaying mortality to high dose of virus. Further, lungs and bronchoalveolar lavage fluid of atopic mice have increased levels of Neuregulin-1 (NRG1), and Nrg1 is highly expressed in CD11c⁺ cells from atopic mice as determined by RNA-seq. Administration of NRG1 protected non-atopic mice from viral induced death. To determine a temporal relationship between NRG1 levels and respiratory viral infection, we utilized an in vitro system of well-differentiated human bronchial epithelial cells and mouse tracheal epithelial cells and show a reduction in viral titer of both RSV and SeV in NRG1 treated cultures. Expression of several genes that have been shown to play a role in airway epithelium integrity and stability were altered by NRG1; potentially regulating viral induced dysregulation of the epithelia and suggesting NRG1 mediated maintenance of homeostasis. In conclusion, our studies demonstrate atopy-induced NRG1 likely plays a novel role in survival from severe respiratory viral infections, and may have therapeutic value to prevent mortality from these infections.

### Results & Discussion

### Pre-Existing Atopy Prevents SeV Induced Lethality

To model the impact of the atopic state in preventing severe disease from respiratory viral infection, we made C57BL6 mice atopic by sensitizing and challenging with house dust mite antigen (HDM (atopic)) or PBS (non-atopic control; NA). We previously demonstrated that inoculating i.n. with 2×10⁵ pfu (“regular” dose) SeV 3 d after the last HDM challenge attenuated weight loss, reduced SeV titer, and inhibited SeV induced airway hyperreactivity. This prevention of post-viral disease required the presence of PMN in the atopic mice (Hussain et al., J Immunol 207:2589-2597, 2021). Given that atopic mice had suppressed respiratory viral induced disease with the regular dose of SeV, we decided to examine the impact of pre-existing atopy on a normally lethal dose of SeV (2×10⁶ pfu; “high” dose). Interestingly, like with IAV infection in mouse models all mice made atopic before SeV infection survived high dose viral inoculation, while all NA mice did not (FIG. 1A). Survival in atopic mice was associated with a significant reduction in viral titer (FIG. 1B).

### CD11c⁺ Cells From Atopic Mice Delay SeV Induced Mortality

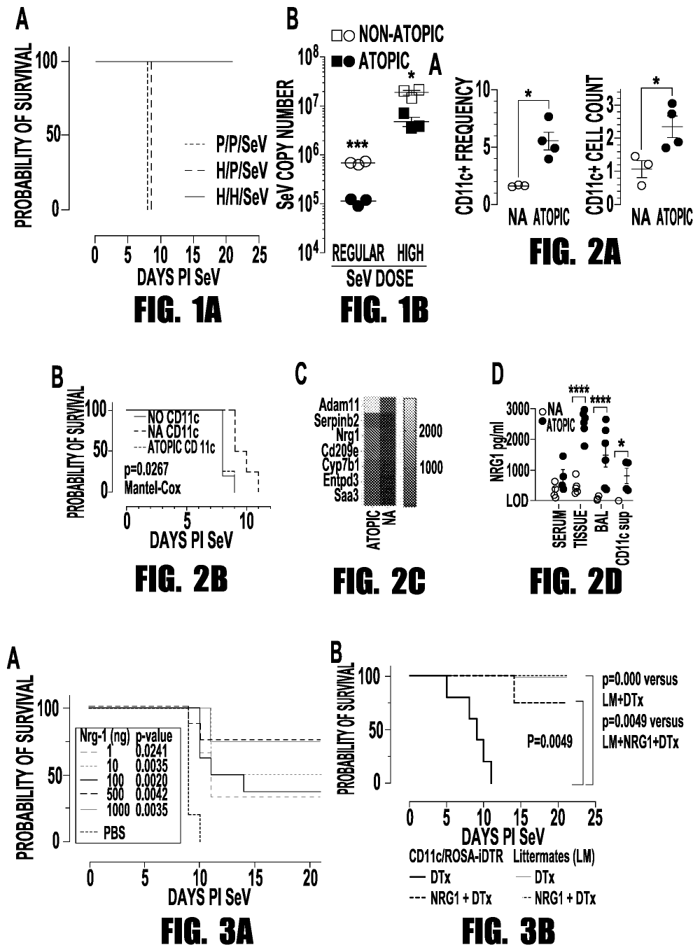

We have shown that in atopic mice the protection from post-viral airway disease depends upon a PMN dependent process related to increased viral uptake by phagocytic PMNs (Hussain et al., 2021, ibid.) however, the protection that atopy provides from mortality did not appear to be due to PMNs, as depletion of PMNs in atopic mice failed to reduce the protection against mortality provided by the atopic status. Since PMNs did not appear to be responsible for the protection against mortality, we examined other cell types. In atopic mice we noted significantly increased frequency and cell numbers of CD11c⁺ cells (dendritic cells or macrophages) in the airways (FIG. 2A). We have previously demonstrated a critical role for CD11c⁺ cells in the immune axis linking SeV to post-viral airway disease (Hussain et al., 2021); (Grayson et al., J Exp Med 204:2759-2769, 2007); (Grayson et al., J Immunol 179:1438-1448, 2007)). Therefore, we posited that protection against lethal SeV infection could be due to CD11c⁺ cells from atopic mice. Depletion of CD11c+ cells is associated with increased mortality to regular dose SeV, so we are unable to determine if depletion of these cells would reverse the protection seen in atopic mice. However, we did isolate CD11c⁺ cells from atopic mouse lung and adoptively transferred them into naïve mice. Transfer of these cells 24 h before inoculation with high dose SeV led to a significant delay in mortality, but did not prevent death (FIG. 2B). These data suggest that CD11c⁺ cells in atopic mice are important in providing the survival advantage. To better understand the requirement for CD11c⁺ cells, we analyzed the trancriptional changes in the CD11c+ cells that occur as a result of making the mice atopic. CD11c⁺ cells were isolated from atopic and NA mouse lung and RNA-seq performed. While there were several dysregulated gene products, one gene product whose expression was increased several-fold in atopic CD11c⁺ cells was neureglin-1 (NRG1; FIG. 2C). NRG1 is a ligand for ErbB receptors and its expression has been shown to be beneficial in coronary heart disease, but potentially detrimental in hepatitis C virus infection. Given the RNA-seq data and rationale, we measured NRG1 protein levels and found them significantly elevated in the lungs and airways of atopic mice, as well as in ex-vivo cultured CD11c⁺ cells isolated from atopic mouse lung (FIG. 2D).

### NRG1 Prevents Death From Respiratory Viral Infection in Vivo and Respiratory Viral Replication in Vitro

Neuregulin 1 (NRG1), a 44-kD glycoprotein, is a cytokine of the epidermal growth factor family and is expressed in 14 isoforms due to alternate splicing or use of alternate promoters. All isoforms contain either an alpha or beta variant epidermal growth factor (EGF)-like domain at its C-terminus that bind to the ErbB receptor tyrosine kinases (ErbB1-ErbB4). The EGF-like motif is sufficient for most biological effects of the full-length ErbB proteins. Appert-Collin et al., Front Pharmacol 6:283, 2015. NRG1 appears to play a homeostatic role with studies showing that loss or overexpression of NRG1 can disrupt NRG1-ErbB signaling (Wang et al., Nat Commun 12:278, 2021); however, little is known about the role of NRG1 in respiratory viral infections. Given the elevated level of NRG1 in the airways of atopic but not NA mice, we explored whether NRG1 alone could protect mice from respiratory viral induced mortality.

To determine if NRG1 contributes to survival, we administered NRG1 (10 ng to 1000 ng daily) i.n for 5 days to naïve mice before infecting with high dose SeV and determining mortality. In a dose responsive fashion, survival was increased with NRG1 administration (FIG. 3A). Interestingly, NRG1 did not fully protect against post-viral airway disease as mucus cell metaplasia (MCM) was increased, while AHR was blunted but not prevented; these data support the contention that the survival mechanism is distinct from that of post-viral airway disease.

As mentioned previously, depletion of CD11c⁺ cells from mice before a SeV infection was associated with a marked increase in mortality. We hypothesized that If NRG1 was coming from CD11c⁺ cells, it is possible that NRG1 is required for survival, even from regular dose SeV. To test this hypothesis we provided NRG1 to CD11c/ROSA-iDTR mice (and littermates) via i.n delivery daily for five days (day −4 to 0) and on day −2 (at the time of the 3rd NRG1 administration) diphtheria toxin (DTx, 15 ng/gm) was delivered i.p. On day 0 mice were inoculated with 2×10⁵ pfu SeV (regular dose) and survival determined. As shown in FIG. 3B, NRG1 was able to significantly reduce the mortality in CD11c depleted mice while control mice without NRG1 succumbed to the viral infection. While intriguing, this experiment does not directly show the CD11c⁺ cells in the mice are making NRG1, but does demonstrate the ability of NRG1 to prevent viral induced death. This protection could be due to replacement of NRG1 that may have come from the depleted CD11c⁺ cells and/or it could have been due to a direct effect on viral replication in epithelial cells.

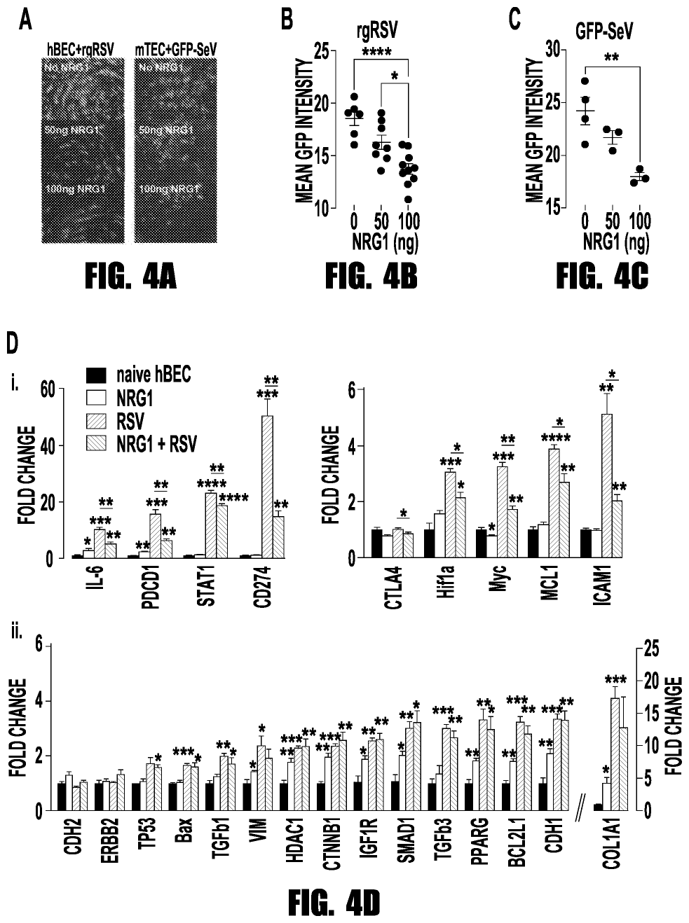

To determine if NRG1 had a direct effect on respiratory viral replication, we utilized in vitro human and mouse airway epithelial cell culture systems. The airway epithelium provides the first line of defense against inhaled bacteria and viruses. Therefore, maintenance and integrity of the bronchial epithelium is critical for its barrier function. Well-differentiated human bronchial epithelial cultures (hBEC) were treated on the basolateral side with NRG1 (10 ng, 50 ng, 100 ng) in 500 μl media 5 and 3 days before, as well as with the viral inoculum of 4000 pfu RSV encoding GFP (rgRSV). As can be seen in FIGS. 4A & B, RSV replication was reduced (i.e., reduced GFP expression) in NRG1 treated hBEC. A similar result was obtained in mouse tracheal epithelial cultures (mTEC) inoculated with GFP-expressing SeV (GFP-SeV) (FIG. 4A & C).

### NRG1 Modulates Homeostasis Related Genes in Epithelial Cells

Upon infection of airway epithelium, respiratory viruses such as RSV and rhinovirus augment chronic inflammation and delay viral clearance while others such as COVID-19, lead to severe damage of the epithelial cell barrier due to cell death. Since NRG1 is required for maintaining epithelial differentiation via ErbB signaling and promotes epithelial cell proliferation and repair (Liu and Kern, Am J Respir Cell Mol Biol 27:306-313, 2002), we examined the effect of NRG1 on cellular proliferation in RSV-infected hBEC. NRG1 pretreatment led to increased thickness of the epithelial cell layer in RSV-infected hBEC suggesting that similar to other epithelial injury models (Tan et al., Front Cell Dev Biol 8:99, 2020) NRG1 may support epithelial proliferation and repair processes in viral infections.

It has been reported that NRG1 plays a critical role in maintaining homeostasis potentially by reducing cellular and mitochondrial stress (Zhang et al., Oxid Med Cell Longev 2016:3849087, 2016). Using a custom gene array and NRG1 treated or untreated RSV-infected hBEC, we evaluated the gene expression associated with the homeostatic/protective function of airway epithelium. As can be seen in FIG. 4D, administration of NRG1 to uninfected epithelial cells had little effect on most gene products examined. However, infection with RSV led to a marked and significant upregulation in many gene products. Importantly, treatment with NRG1 reduced expression of eight of those gene products-IL6, HIF1α, MYC, MCL1, ICAM-1, PDCD1, STAT1 and PD-L1 (FIG. 4D(i)). Further studies are needed to determine functional co-relationships between these genes and their protein products; however, we examined the expression of IL6, which has both pro-and anti-inflammatory functions. IL6 was increased by RSV infection of hBEC; however, NRG1 treatment of epithelial cells significantly attenuated this upregulation of IL6 expression 48 h PI RSV (FIG. 4D(i) left panel) supporting a role of NRG1 in regulation of inflammatory responses.

PD-1, encoded by PDCD1 mitigates inflammatory cell activation and also contributes to mitochondrial dysfunction, while its ligand, PD-L1, has increased expression on lung epithelial cells. Thus, potentially by impairing expression of both PD-1 and PD-L1, NRG1 could increase the antiviral inflammatory response leading to increased survival. Supporting this hypothesis is an in vitro hBEC study that demonstrated blocking PD-L1 in acute RSV infection facilitated the antiviral immune response (Telcian et al., J Infect Dis 203:85-94, 2011). Downregulation of the PD-L1-PD1 axis by NRG1 further suggests a protective role in acute respiratory infection.

Some viruses including RSV delay apoptosis of infected cells to prevent premature death of host cells by increasing anti-apoptotic, MCL-1 expression to promote viral latency and persistent infection. Our data supports RSV infection increasing MCL1 with a significant reduction in MCL1 expression in hBEC pre-treated with NRG1 prior to RSV infection; however, MCL1 is still significantly higher in the NRG1+RSV group than in cells treated with NRG1 without RSV infection (FIG. 4D(i), right panel). These data suggest that NRG1 may induce controlled (or selective) apoptosis in infected cells as a means of protection from the viral insult. Further, caspase-3, an apoptotic effector caspase, has been shown to cleave MCL-1 thus facilitating apoptosis (Weng et al., J Biol Chem 280:10491-10500, 2005)). Although we have not looked at the expression of MCL-1 protein in mouse lung epithelium, we did find increased activated caspase-3 at day 5 PI SeV in small airways of NRG1 treated mice. The increased levels of active caspase-3 suggests that NRG1 in mice induces a similar pro-apoptotic state as we see in the hBEC.

ICAM-1 has been shown to facilitate RSV entry and infection of epithelial cells (Behera et al., Biochem Biophys Res Commun 280:188-195 (2001)); therefore, reduced expression of ICAM-1 in NRG1 treated RSV infected hBEC could contribute to reduced virus spreading. Moreover, ICAM-1 expression is still significantly higher in NRG1 treated and infected hBEC than in untreated or uninfected NRG1 treated epithelial cells suggesting a potential role in tissue repair and resolving inflammation (FIG. 4C), (Bui et al., J Leukoc Biol 108:787-799, 2020). MYC activation has been shown to provide support for respiratory virus replication including IAV and adenovirus (Thai et al., Nat Commun 6:8873 (2015)). Therefore, a reduction in MYC expression in our study further suggests NRG1 could be inhibiting RSV replication (FIG. 4D(i)). HIF1α, a transcription factor, has been implicated in viral infections (VSV, HSV) and in the cytokine storm seen with severe COVID-19 disease (Tian et al., Signal Transduct Target Ther 6:308, 2021). Increased HIF1α is often a consequence of mitochondrial damage, which will impair epithelial repair similar to what we have reported with mouse Hif1α and re-epithelialization of skin wounds. Therefore, a reduction in HIF1α expression in hBEC with NRG1 and RSV further supports the notion that NRG1 is a homeostatic regulator of viral infection (FIG. 4D(i)). Finally, there were genes with small but significant increases in expression in NRG1 treated hBEC relative to naïve cells (FIG. 4D(ii)). Altogether, we posit that these data suggest NRG1 reduces the cellular stress imposed by RSV on infected hBEC, while at the same time increasing apoptosis of these cultured cells.

In conclusion, our studies demonstrate that the presence of atopy before a severe paramyxoviral infection is sufficient to protect against an otherwise lethal infection. This protection appears to be driven by CD11c⁺ cells from atopic mouse lung that express NRG1. Intranasal administration of NRG1 is sufficient to protect mice from a lethal viral infection, and administration of NRG1 to epithelial cell cultures ex vivo is sufficient to reduce RSV (for human epithelial cells) and SeV (for mouse epithelial cells) replication. This reduced viral titer is associated with a decrease in expression of the PD-1, PD-L1 axis. Together, we have identified a novel role for the ErbB ligand NRG1 that provides protection from a severe viral infection. Future studies will explore in more detail the mechanism of NRG1 mediated protection, as well as the therapeutic potential of NRG1 for the treatment of severe respiratory viral infections.

### Materials & Methods

### Animals

C57BL/6 (wild type) mice, C57BL/6-Gt (ROSA) 26Sor^{tm1(HBEGF)Awai}/J (common name: ROSA26iDTR (Strain #: 007900)) and C57BL/6J-Tg (Itgax-cre,-EGFP) 4097Ach/J (common name: CD11c-Cre-GFP line 4097 (strain #: 007567)) were purchased from the Jackson Laboratory (Bar Harbor, Maine, USA) and bred in-house. Unless indicated otherwise mice 8-12 weeks old were used for the experiments.

### Neuregulin-1 Protein

For mouse studies, Recombinant Mouse Neuregulin-1/NRG1 Protein, CF (from R&D Systems (cat #: 9875-NR-050)) was used. The NRG1 protein was obtained from Accession number NP_001351350, which is a pro-neuregulin protein sequence for isoform 2. The accession number for the source NRGlapha mRNA transcript is NM_001364421.1. For human cell culture studies we have used NRG1 alpha form however, NRG1beta has been shown to have greater efficacy in neuronal system.

### Human and Mouse Air-Liquid Interphase Culture

Human bronchial epithelial cultures (hBEC) progenitors and C57BL6 mouse tracheal epithelial cultures (mTEC) were grown on transwells at the air-liquid interface for 5 to 6 weeks for the development of pseudostratified well-differentiated airway epithelial layers resembling in vivo epithelium with ciliated, goblet and basal cell types as we have published (Hussain et al., J Immunol 207:2589-2597, 2021).

### Atopic Model and SeV Infection

C57BL6 mice were sensitized with 1 μg and challenged with 10 μg of HDM extract (catalog no. XPB91D3A2.5; Stallergenes Greer USA, Boston, MA) given intranasally (i.n.) to make them atopic or with PBS as non-atopic (NA) control. Three days after last challenge mice were inoculated i.n. with 2×10⁵ pfu (regular dose) or 2×10⁶ (high dose) SeV.

### Flow Cytometry and Flow-Activated Cell Sorting (FACS) of CD11c⁺ Cells

Cells from mouse whole lungs were harvested and flow cytometry and FACS were performed as we previously reported using standard cell staining techniques using CD11c antibody (Clone N418; catalog no. 12-0114-82, ThermoFisher) or Armenian Hamster IgG Isotype Control (Clone: eBio299Arm, catalog no. 12-4888-81, ThermoFisher).

Adoptive transfer and SeV infection

Lung CD11c⁺ cells purified (≥85% CD11c⁺) from NA and atopic mice by FACS were delivered intranasally to anesthetized naïve C57BL6 mice at a concentration of 1.0×10⁶ cells/mouse. After 24 hrs recipient mice were inoculated with 2.0×10⁶ pfu SeV and survival recorded over days post inoculation.

### Inducible Depletion of CD11c, NRG1 Treatment and SeV Infection

In order to have mice with diphtheria toxin (DTx) inducible depletion of CD11c we crossed CD11c-Cre-GFP with ROSA26iDTR and progeny was named CD11c/ROSA-iDTR. Littermates and CD11c/ROSA-iDTR were treated with or without NRG1 (500 ng) daily for five days (d−4 to d0) and on d−2 DTx (15 ng/gm) was delivered i.p before inoculation with regular dose of SeV at day 0. Mortality was then determined.

### Airway Hyper-Reactivity (AHR) and Mucus Cell Measurement

Invasive measurement of AHR was performed by measuring airway resistance to increasing doses of methacholine using the FlexiVent system as we have published (Hussain et al., 2021, Ibid.). Mucus cell metaplasia was determined by performing Periodic-Acid-Schiff (PAS) staining of formalin fixed lung sections and in a blinded fashion, manually counting and determining the number of PAS⁺ cells per mm of basement membrane (using ImageJ) as we have reported (Hussain et al., 2021, Ibid.).

### Immunohistochemistry

Paraffin embedded mouse lung tissues sectioned at 10 μM thickness were dewaxed followed by heat induced antigen retrieval and stained with active caspase-3 antibody (catalog #: AF835-SP, R & D Systems) at 1 μg/ml overnight at 4° C. followed by incubation with the Anti-Rabbit IgG VisUCyte™ HRP Polymer Antibody (catalog #VC003, R & D Systems). Tissues were stained with DAB (brown) and counterstained with hematoxylin (blue) following manufacturer's instructions.

### RNA Sequencing

Whole transcriptome profiling was performed by preparing strand-specific RNA-seq libraries using NEB Next Ultra II Directional RNA Library Prep Kit for Illumina, following the manufacturer's recommendations. In summary, total RNA was assessed using RNA 6000 Nano kit on Agilent 2100 Bioanalyzer (Agilent Biotechnologies) and Qubit RNA HS assay kit (Life Technologies). A 140-500 ng aliquot of total RNA was rRNA depleted using NEB's Human/Mouse/Rat RNAse-H based Depletion kit (New England BioLabs). Following IRNA removal, mRNA was fragmented and then used for first-and second-strand cDNA synthesis with random hexamer primers and ds cDNA fragments undergoing end-repair and a-tailing and ligation to dual-unique adapters (Integrated DNA Technologies). Adaptor-ligated cDNA was amplified by limit-cycle PCR. Library quality was analyzed on Tapestation High-Sensitivity D1000 ScreenTape (Agilent Biotechnologies) and quantified by KAPA qPCR (KAPA BioSystems). Libraries were pooled and sequenced at 2×150 bp read lengths on the Illumina HiSeq 4000 platform to generate approximately 60-80 million paired-end reads per sample. Differential expression analysis was performed and significant differentially expressed features between the two groups with an absolute value of fold change≥1.5 and an adjusted p-value of ≤0.10 (10% FDR) were recorded.

### NRG1 ELISA

Detection of NRG1 levels was performed using mouse NRG1 ELISA kit (cat #EKN47308, Biomatik, Delaware USA) following manufacturer's instruction. Freshly prepared standards, tissue extract and cell supernatant were used for the assay and results quantified by plotting against the standard curve with detection range of 15.6-1000 pg/ml and reading O.D. at 450 nm.

### NRG1 Exogenous Administration and Cell Culture Treatment

Recombinant mouse neuregulin-1/NRG1 protein, CF (cat #: 9875-NR) (Novus Biologicals, CO, USA) of varying concentrations (10 to 500 ng) were given i.n. daily for 5 days before inoculating with SeV. Data were recorded for weight change and survival for two weeks post infection. Cell culture basal media was supplemented with recombinant human NRG1 alpha (cat #: NBP2-35093, Novus Bio) for hBEC or with mouse NRG1 for mTEC on day five and three before and at the time of infection with 4000 pfu of rgRSV or SeV-GFP. Virus inoculation occurred in 100 μl DMEM on apical side for 4 hrs at 37° C. in 5% CO₂ incubator. GFP levels were quantified by imaging with EVOS cell imaging system at 10× magnification and determining the mean fluorescent intensity with ImageJ software. Cell culture harvested after 48 h for RNA isolation and qRT-PCR. The SeV-GFP was a gift from Dr. Dominique Garcin, Switzerland and the construct is published (Strahle et al., J Virol 81:12227-12237, 2007) and rgRSV, that also expresses GFP, was developed by our group (Kwilas et al., J Virol 84:7770-7781, 2010).

### RNA Isolation and Quantitative Real-Time Polymerase Chain Reaction (PCR)

Total RNA from hBEC isolated using mirVana miRNA and total RNA isolation kit (catalog no. AM1560, Thermo Fisher Scientific). For qRT-PCR cDNA was synthesized using Maxima H Minus cDNA synthesis kit (catalog no. M1681; Thermo Fisher Scientific). PrimePCR™ PCR Primers assay plates were custom made with validated primers for use with EvaGreen dye-based chemistry (Bio-RAD, USA). Samples were run on CFX96 Touch Real-Time Detection System (Bio-RAD, USA). CT value normalized with GAPDH or MRPL13 and expressed as fold change relative to naïve control. For the array we selected 24 genes that broadly fall into three groups: i) epithelial to mesenchymal transition signature genes; (ii) genes in RNA virus infection panel of pre-designed human PrimePCR by BioRad; (iii) fibroblast genes previously reported to be regulated by NRG1 (De Keulenaer et al., Circ Heart Fail 12: e006288, 2019).

Following human genes were tested in the assay: BAX (BCL2-associated X protein); BCL2L1 (BCL2-like 1); CD274 (PD-L1); CDH1 (cadherin 1, type 1, E-cadherin (epithelial); CDH2 (cadherin 2, type 1, N-cadherin (neuronal); COL1A1 (collagen, type I, alpha 1); CTLA4 (ICOS, cytotoxic T-lymphocyte-associated protein 4); CTNNB1 (catenin (cadherin-associated protein), beta 1, 88 kDa), GAPDH (glyceraldehyde-3-phosphate dehydrogenase); HDAC1 (histone deacetylase 1); H1F1A (hypoxia inducible factor 1); ICAM1 (intercellular adhesion molecule 1); IGF1R (insulin-like growth factor 1 receptor); IL6 (interleukin 6); MCL1 (myeloid cell leukemia sequence 1 (BCL2-related)); MRPL13 (mitochondrial ribosomal protein L13); MYC (v-myc myelocytomatosis viral oncogene homolog (avian); PDCD1 (programmed cell death 1); PPARG (peroxisome proliferator-activated receptor gamma); SMAD1 (SMAD family member 1); STAT1 (signal transducer and activator of transcription 1, 91 kDa); TGFB1 (transforming growth factor, beta 1); TGFB3 (transforming growth factor, beta 3); TP53 (tumor protein p53); VIM (vimentin)

### Statistical Analysis

All statistical analyses were performed using Prism 9 (GraphPad Software Inc.). All normally distributed data are presented as mean±SEM with survival analyses using Kaplan-Meier curves and log-rank (Mantel-Cox) tests. Student's t test (for parametric data) was used to assess significant differences between two means. For all tests, *, P<0.05; **, P<0.001; *** was considered statistically significant.

### Example 2: Role of Alarmins in NRG1 Production

Using house dust mite (HDM) to make C57BL6 mice atopic, the inventors found that pre-existing atopy protected mice from developing post-viral airway hyperreactivity and mucous cell metaplasia (post-viral airway disease). This protection required increased phagocytosis of viral particles by lung neutrophils (PMN) in an apparent IL4 dependent process. They also demonstrate that pre-existing atopy prevents death from an otherwise lethal dose of SeV, and this protection is dependent in part upon IL33. Using depletion and adoptive transfer experiments, the inventors have also demonstrated that airway CD11c⁺ cells (likely dendritic cells or macrophages), but not PMNs, are required for increased survival. Further, IL33 and TSLP induce production of neuregulin-1 (NRG1) in murine CD11c⁺ cells and human CD14+ monocytes, and exogenous administration of NRG1 is sufficient to prevent mortality caused by SeV infection. The inventors have also demonstrated that in vitro culture of airway epithelial cells (human and mouse) with NRG1 significantly impairs the ability of both SeV and RSV to replicate in airway cells from mouse and human, respectively.

Based upon these data, the inventors proposed the hypothesis that pre-existing atopy protects against respiratory viral induced mortality in an IL33 and/or TSLP dependent process that drives CD11c⁺ cells to produce NRG1, which protects epithelial cells from viral infection.

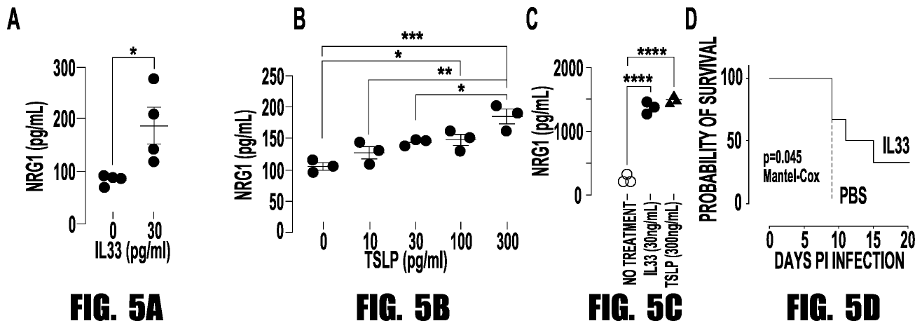

The inventors also hypothesize that IL33 and/or TSLP directly acts upon specific mouse CD11c or human CD14 expressing cells to induce NRG1 production and this leads to protection from lethality. Culturing mouse CD11c⁺ cells or human CD14⁺ monocytes with IL33 or TSLP was found to significantly increase NRG1 production by these cells, as shown in FIGS. 5A-5D. Addition of NRG1 to epithelial cell culture reduced viral replication and reduced inflammatory gene product production by RSV infected epithelial cells, suggesting restoration of homeostasis in these cells (FIGS. 4A-D). The results demonstrate that NRG1 directly acts upon epithelial cells to protect them from a viral insult and to limit RSV and SeV replication.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of treating or decreasing the risk of developing a respiratory viral infection in a subject by administering a therapeutically effective amount of neuregulin to the subject.

2. The method of claim 1, wherein the neuregulin is neuregulin-1.

3. The method of claim 1, wherein the method treats a respiratory viral infection.

4. The method of claim 1, wherein the respiratory viral infection is an influenza, coronavirus, rhinovirus, metapneumovirus, respiratory syncytial virus or parainfluenza virus infection.

5. The method of claim 1, wherein the respiratory viral infection is a respiratory syncytial virus or parainfluenza virus infection.

6. The method of claim 1, wherein the neuregulin is administered by pulmonary administration.

7. The method of claim 1, wherein the neuregulin is administered as an aerosol.

8. The method of claim 1, wherein the subject is human.

9. A method of decreasing the risk a subject will develop post-viral airway disease by administering an effective amount of neuregulin to the subject.

10. The method of claim 9, wherein the neuregulin is neuregulin-1.

11. The method of claim 9, wherein the subject has a respiratory virus infection.

12. The method of claim 11, wherein the respiratory viral infection is an influenza, coronavirus, rhinovirus, metapneumovirus, respiratory syncytial virus or parainfluenza virus infection.

13. The method of claim 11, wherein the respiratory virus infection is a respiratory syncytial virus or parainfluenza virus infection.

14. The method of claim 9, wherein the subject has an increased risk of developing a respiratory virus infection.

15. The method of claim 9, wherein the subject is a human subject.

16. The method of claim 9, wherein the post-viral airway disease is asthma.

17. The method of claim 9, wherein the subject is human.

18. The method of claim 9, wherein the neuregulin is administered by pulmonary administration.

19. The method of claim 9, wherein the neuregulin is administered intra nasally.

20. The method of claim 9, wherein the neuregulin is administered as an aerosol.


SEQUENCE LISTING (1 sequence)

SEQ ID NO: 1 - protein, 64 aa, Homo sapiens
SHLVKCAEKEKTFCVNGGECFMVKDLSNPSRYLCKQPGFTGARCTENVPMKVQNQEKAEELYQK